(19) United States
(12) Patent Application Publication
(10) Pub. No.: US 2016/0158155 A1
(43) Pub. Date: Jun. 9, 2016

(54) MAGNETIC NANOPARTICLES USEFUL FOR MAGNETIC SENSOR DETECTION ESPECIALLY IN BIOSENSOR APPLICATIONS

(71) Applicants: Makoto Murakami, Ann Arbor, MI (US); Bing Liu, Ann Arbor, MI (US)

(72) Inventors: Makoto Murakami, Ann Arbor, MI (US); Bing Liu, Ann Arbor, MI (US)

(21) Appl. No.: 14/227,277

(22) Filed: Mar. 27, 2014

Related U.S. Application Data
(60) Provisional application No. 61/805,539, filed on Mar. 27, 2013.
(60) Provisional application No. 61/933,989, filed on Jan. 31, 2014.

(51) Int. Cl. A61K 9/16; G01N 33/58; A61K 49/18

(52) U.S. Cl. CPC A61K 9/1617; A61K 49/1839; G01N 33/58; Y10S 977/773; Y10S 977/932; B82Y 25/00

(57) ABSTRACT

Disclosed is process for preparing magnetic nanoparticles (MNPs) that results in very sensitive MNPs that can be used in a variety of diagnostic and analytical methods. The MNPs exhibit superparamagnetism and find special use in giant magnetoresistance sensors (GMRS). The MNPs are created by a process that permits one to tune the size of nanoparticles to a range of from 10 to 20 nanometers with a very small particle size distribution of +/−2 nanometers or less. The MNPs can be tagged with a variety of markers and thus find use in many analytical assays, cell sorting techniques, imaging methods, drug delivery methods and cancer treatments. The inventive MNPs can be detected in magnetic file strengths of 2000 Oe or less.

### RELATED APPLICATIONS

This application claims the benefit of U.S. Provisional Application No. 61/805,539 filed on Mar. 27, 2013 and US Provisional Application No. 61/933,989 filed on Jan. 31, 2014.

### STATEMENT REGARDING FEDERALLY SPONSORED RESEARCH

NONE.

### TECHNICAL FIELD

This invention relates generally to magnetic nanoparticles and more particularly to magnetic nanoparticles that are superparamagnetic.

### BACKGROUND OF THE INVENTION

Magnetic nanoparticles are known in the art, for example: MACS® from Miltenyi Biotec for magnetic-assisted cell sorting applications; Dynabeads® from Life Technologies; and magnetic nanoparticles from Ocean Nanotech. They have found use in magnetic-assisted cell sorting, isolation of proteins, isolation of RNA and DNA segments, immunoassays and other diagnostic procedures. The magnetic nanoparticles are also used as tags for biological sensor applications using magnetic field sensor devices such as Giant Magnetoresistance (GMR) and Tunnel Magnetoresistance (TMR) sensor devices. Construction and uses of GMR sensors are described in the literature, for example see, “Giant Magnetoresistive Biosensors for Molecular Diagnosis: Surface Chemistry and Assay Development”, Heng Yu et al., Proc. of SPIE Vol. 7035, 70350E (2008); “The Matrix Neutralized”, Ilia Fishbein and Robert J. Levy, Nature, Vol 461, 890, (15 Oct. 2009); and “Giant Magnetoresistive Biochip for DNA Detection and HPV Genotyping”, Liang Xu et al., Biosensors and Bioelectronics 24, 99, (2008).

Magnetic nanoparticles (MNPs) exhibit superparamagnetism when they are in the size range of from about 3 nanometers (nm) to about 50 nm and this property has been used to create cell sorting methods, bioanalytical assays and forms of Giant Magnetoresistance Sensors (GMRS).

It is desirable to provide MNPs having a higher magnetic sensor response than those currently available. This will allow for detection of lower levels of analytes and improved sensitivity. This technology may have application in creating higher sensitivity responses in systems that use GMRS devices.

### SUMMARY OF THE INVENTION

In general terms, this invention provides magnetic nanoparticles (MNPs) with a very small size distribution and very high AC susceptibility that can be used in a variety of applications.

By way of example, one method for creating MNPs according to the present invention can comprise the steps of: mixing iron acetylacetonate Fe(acac)3 1,2 hexadecanediol, oleic acid, oleylamine, in a volume of trioctylamine under a blanket of a non-reactive gas with heating to 120° C. for 1 hour, wherein the molar ratio of oleic acid and the molar ratio of oleylamine to the molar level of iron acetylacetonate are independently from 1:1 to 2.5:1; heating the mixture to 200° C. for 2 hours; then heating the mixture up at a rate of 2° C./minute to a reflux temperature of 350° C. and refluxing for 2 hours; then cooling the mixture to room temperature by removal of the heat source; then precipitating the magnetic nanoparticles by adding ethanol to the mixture and recovering them via centrifugation.

In another embodiment, the present invention comprises water dispersible MNPs such as iron oxide magnetic nanoparticles having a particle size of from 10 to 25 nanometers (nm), preferably 13 to 20 nm and most preferably 13 to 18 nanometers with a size distribution of +/−2 nanometers and having a surfactant coating of oleic acid and oleylamine wherein, independently, the molar ratio of oleic acid and the molar ratio of oleylamine to iron oxide are from 1:1 to 2.5:1.

In another embodiment, the present invention comprises MNPs having an AC susceptibility per particle in a liquid state at 25° C. that satisfies the following formula:

χ/N≧A(D−13)

Wherein: χ is the AC susceptibility at 100 Hz
N is the number of MNPs
D is the diameter of the nanoparticles in nanometers
A equals

$$\frac{( χ / N )}{D}$$

and ranges in value from 5*10⁻¹⁷ to 2*10⁻¹⁶
   In the present specification and claims the term “in a liquid state” means the magnetic nanoparticles are suspended in a liquid. The preferred liquid is water; however the measured AC susceptibility will be similar in other liquids having a viscosity that is similar to that of water. For purposes of example, other liquids having a sufficiently similar viscosity to water include: phosphate buffered saline, biological buffers, chloroform, toluene, hexane, methanol, and ethanol. If one suspends the magnetic nanoparticles in very high viscosity liquids then there will be a decrease in the AC susceptibility, but this is expected to occur only in very high viscosity liquids.

These and other features and advantages of this invention will become more apparent to those skilled in the art from the detailed description of a preferred embodiment. The drawings that accompany the detailed description are described below.

### BRIEF DESCRIPTION OF THE DRAWINGS

FIG. 1A is a representation of a size distribution for a magnetic nanoparticle have a size of 15 nm+/−0.5 nm, FIG. 1B is a plot of the theoretical values for χ_{—real}, top trace, and χ_imag, bottom trace, for the magnetic nanoparticle of FIG. 1A;

FIG. 2A is a representation of a size distribution for a magnetic nanoparticle have a size of 15 nm+/−2.0 nm, FIG. 2B is a plot of the theoretical values for χ_{—real}, top trace, and χ_imag, bottom trace, for the magnetic nanoparticle of FIG. 2A;

FIG. 3A is a representation of a size distribution for a magnetic nanoparticle have a size of 15 nm+/−5.0 nm, FIG. 3B is a plot of the theoretical values for χ_{—real}, top trace, and χ_imag, bottom trace, for the magnetic nanoparticle of FIG. 1A;

FIG. 4A is a plot of the theoretical value for χ_{—real} versus peak particle size, assuming a size distribution of +/−1 nm, after normalization to a frequency of 100 Hz at 25° C.;

FIG. 4B is a plot of the theoretical value for χ_{—real} calculated assuming a peak particle size of 15 nm and varying the size distribution from +/−0.5 to +/−5 nm with normalization to 100 Hz and 25° C.;

FIG. 5 is a schematic of a process for creating magnetic nanoparticles according to the present invention;

FIG. 6A contains a series of plots of the Giant Magnetoresistive Sensor (GMRS) signal obtained versus exposure time from streptavidin labeled magnetic nanoparticles prepared according to the present invention after exposure to a GMRS coated with a series of analyte solutions containing from 0.0001 to 1.0 mg/ml of biotin;

FIG. 6B contains a series of plots of the GMRS signal obtained versus exposure time from commercially available MACS® SA, streptavidin labeled magnetic nanoparticles, after exposure to a GMRS coated with a series of analyte solutions containing from 0.0001 to 1.0 mg/ml of biotin;

FIG. 7 contains a series of plots of the GMRS signal obtained versus exposure time from commercially available MACS® AB, biotin antibody labeled magnetic nanoparticles, after exposure to a GMRS coated with a series of analyte solutions containing from 0.0001 to 1.0 mg/ml of biotin;

FIGS. 8A-D show scanning electron microscopy (SEM) photographs of some of the results shown in FIGS. 6A and FIG. 7, specifically, FIG. 8A and FIG. 8C show the results from sample 4 of MNPs prepared according to the present invention at levels of 1 mg/ml of analyte and 0.0005 mg/ml of analyte, respectively while FIG. 8B and FIG. 8D show the results from MACS® AB magnetic nanoparticles at levels of 1 mg/ml of analyte and 0.0005 mg/ml of analyte, respectively;

FIG. 9 is a schematic diagram showing some uses of the magnetic nanoparticles prepared according to the present invention;

FIGS. 10A-10C show the AC magnetic susceptibility and relative signal strength of magnetic nanoparticles prepared according to the present invention in a liquid state compared to commercially available magnetic nanoparticles in the same liquid state;

FIGS. 11A-11F show the crystallinity as seen in transmission electron microscopy (TEM) photographs and Selective Area Diffraction (SAD) patterns of magnetic nanoparticles prepared according to the present invention compared to commercially available magnetic nanoparticles; and

FIG. 12 is a graph of the hysteresis loop for magnetic nanoparticles prepared according to the present invention.

### DETAILED DESCRIPTION OF A PREFERRED EMBODIMENT

The present invention is directed toward creation of magnetic nanoparticles (MNPs) and their use in diagnostic and medical applications. The currently commercially available MNPs have adequate signal to noise ratios, but one is always seeking to improve signal strength to push the limits of detection lower. The present inventors have created MNPs that have a much improved sensitivity and signal to noise ratio. These MNPs can be tagged with useful reporter tags such as streptavidin. The causes of the improved sensitivity and signal to noise ratio are not currently well understood; however, they seem to be related to a number of unique characteristics of MNPs prepared according to the present invention. These characteristics include much tighter particle size control of the MNPs, lower levels of surfactant on the surface of the MNPs, and a higher degree of crystallinity of the MNPs. The combination of these characteristics produces MNPs that have a defined single magnetic domain structure, have a much higher signal to noise ratio and have enhanced sensitivity to an external magnetic field. Because of these changes the current MNPs are very useful in a variety of roles as a magnetic reporter material for many sensitive magnetic field sensing assays and environments. The present MNPs can be detected at a level of external magnetic field that is much reduced from those previously required to detect magnetic nanoparticles.

The MNPs of the present invention have a theoretical size range of from 10 to 30 nanometers. They are based on magnetic substances such as iron, iron oxides including Fe₃O₄, which is also known as magnetite, and other known magnetic materials, for example, iron ferrite, magnetite, maghemite or a mixture thereof. Other magnetic materials also include those containing nickel, cobalt, alloys of these metals, and some rare earth metals. The current MNPs can be prepared from ferromagnetic materials, ferrimagnetic materials and superparamagnetic materials. The ferrimagnetic and ferromagnetic materials are spontaneously magnetic so long as their temperature is below their Curie temperature; above their Curie temperature they are paramagnetic, meaning they have no magnetic order. Ferrimagnetic materials have high resistivity and anisotropic properties.

Magnetic field sensors such as GMR and TMR are based on a quantum mechanical magnetoresistance effect observed in thin-film laminates composed of alternating layers of ferromagnetic and non-magnetic layers. The non-magnetic layer is used as a non-magnetic conductive layer for GMR and as a non-magnetic insulator layer for TMR. The key effect that is observed is a much larger than expected change, in fact a giant change, in electrical resistance that results from whether the magnetization of adjacent ferromagnetic layers are in a parallel or antiparallel alignment. The overall resistance is low when the layers are in parallel alignment and relatively high when they are in antiparallel alignment. Magnetoresistance is dependence of the electrical resistance of a material on the strength of an external magnetic field. In magnetoresistive devices the observed change in resistance based on a magnetic field is much greater than the anisotropic magnetoresistance, which is typically only a few percent. The magnetization direction can be controlled by application of an external magnetic field. The GMR effect is used to create magnetic field sensors which are used to read data on hard disk drives, to make biosensors, and in microelectromechanical systems. Binding of a MNP to the surface of a GMRS can cause a change in magnetism which is detected as a change in the resistance of the GMRS. Due to the sensitivity of the GMRS very few MNPs have to bind to cause a detectable change in resistance.

Another field where these MNPs can find use is in the creation of cell sorting technologies as described above. The present MNPs can be surface modified to carry an antibody or functional compound that can bind to an antigen on a cell surface or another cell marker, respectively. This makes cells with the antigen or cell marker carry a magnetic charge. The cells of interest can be isolated by pouring a mixture of labeled and unlabeled cells through a magnetic column. The cells tagged with the MNPs are retained in the magnetized column and the other cells are washed through. The tagged cells can then be released by removing the magnetic field. The company Miltenyi Biotec sells the MACS® (magnetic-assisted cell sorting) kits and magnetic sorting columns. The techniques can be used to carry out direct magnetic labeling wherein the MNPs directly bind to an antigen on a cell surface or indirect magnetic labeling wherein the cells are first labeled with a primary antibody and then the MNPs bind to the antibody or a functional group attached to the antibody. Once labeled, cells can be isolated by a variety of means including positive selection of magnetically labeled cells, depletion of unwanted cells by magnetically labeling them, depletion followed by positive selection, or two subsequent positive selections. These techniques have been well developed by Miltenyi Biotec and others and are known to those of skill in the art.

Because of their size the MNPs of the present invention exhibit superparamagnetism. Superparamagnetism is a form of magnetism that appears in small ferromagnetic and ferrimagnetic nanoparticles. In this form of magnetism the magnetization randomly flips direction under the influence of temperature. The time between two flips is called the Néel relaxation time. Normally a ferrimagnetic or ferromagnetic material undergoes a transition to a paramagnetic state, where it has no net magnetization in the absence of a magnetic field, at a temperature above its Curie temperature; for superparamagnetic materials this occurs at a temperature below the Curie temperature. For the superparamagnetism to occur the MNP size has to be sufficiently small enough that the particles are single-domain meaning the particle is a single magnetic domain. The typical size range is in the range of from 3 to 50 nm depending on the material.

The first effect observed for the MNPs created according to the present invention was that the average size and size distribution were critical in achieving a high level of signal. Specifically, the MNPS are preferably from 10 to 25 nm in diameter, more preferably from 13 to 20 nm in diameter and most preferably 13 to 18 nm in diameter. Using MNPs according to the present invention at a size of 15 nm+/−1 nanometer a series of calculations were carried out. In a series of calculations based on an equation relating the DC magnetic susceptibility to the AC magnetic susceptibility, formula 1 below, and the Néel-Arrhenius equation, formula 2 below, one can see the effect of the size and size distribution on the responsiveness of the present MNPs.

$$χ = \frac{{χ}_{0}}{1 + iϖτ} \qquad FORMULA 1$$

Wherein: χ is the AC magnetic susceptibility
χ₀ is the DC magnetic susceptibility
i is the square root of −1
ω is the frequency
τ is the relaxation time, which can be seen as response time

τ_{N}=τ₀ exp(KV/k_{B}T) FORMULA 2

Wherein: τ_{N} is the Néel relaxation time
τ₀ is a material dependent time constant called the attempt time or attempt frequency and it has a typical value of from 10⁻⁹ to 10⁻¹⁰ second
K is a material dependent constant, the magneto anisotropy energy density of the nanoparticle
V is the nanoparticle volume
k_{B} is the Boltzmann constant
T is the temperature

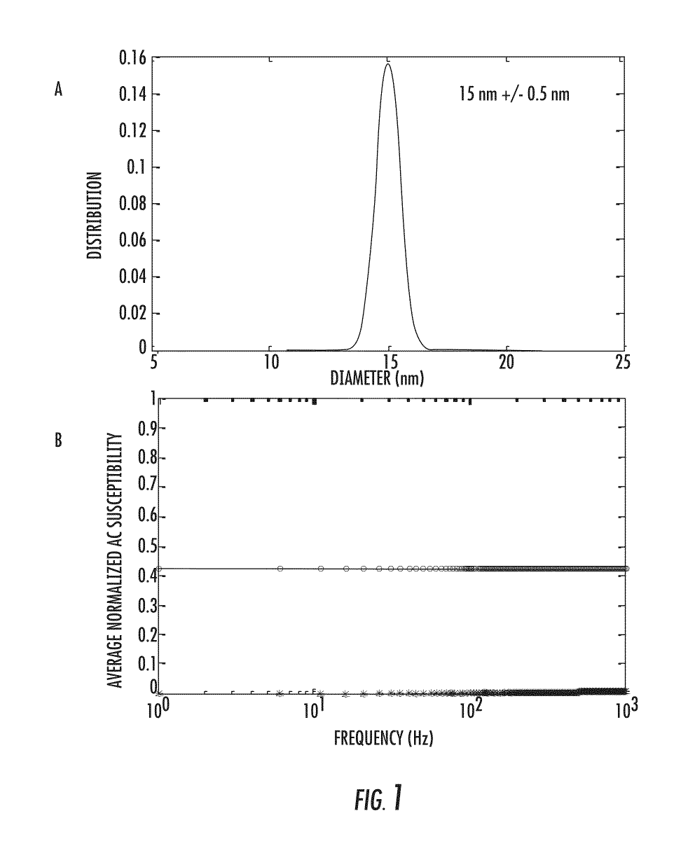

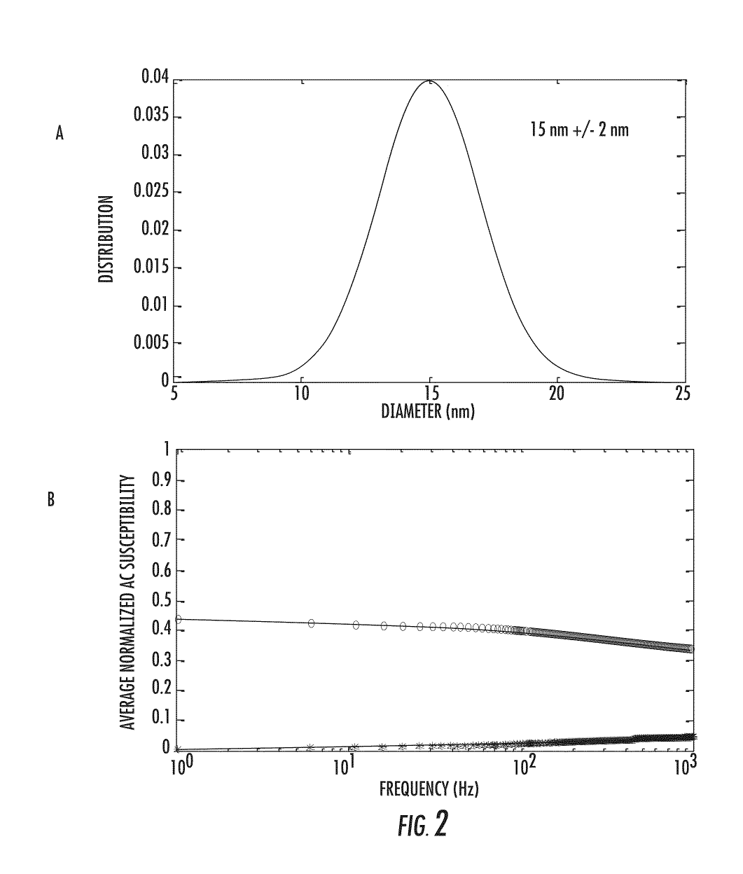

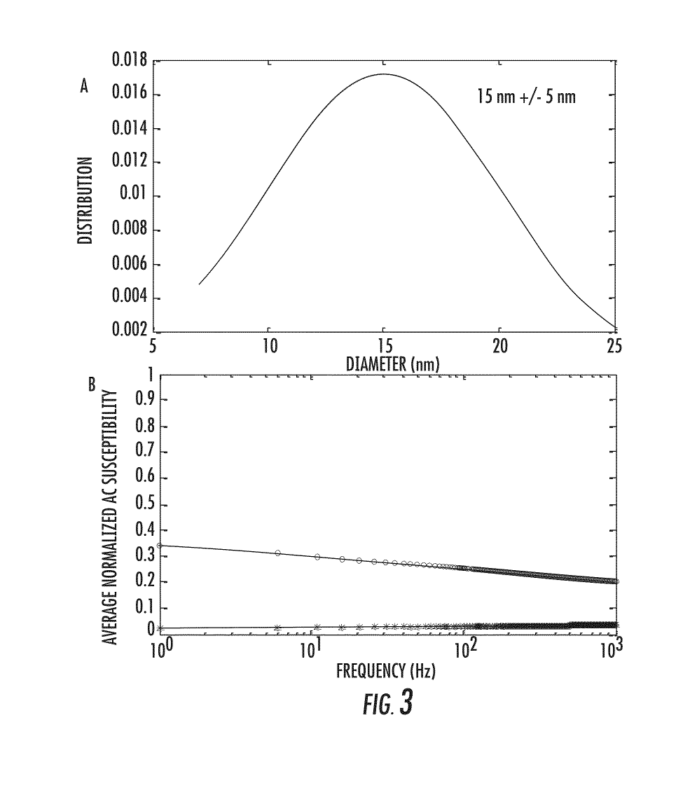

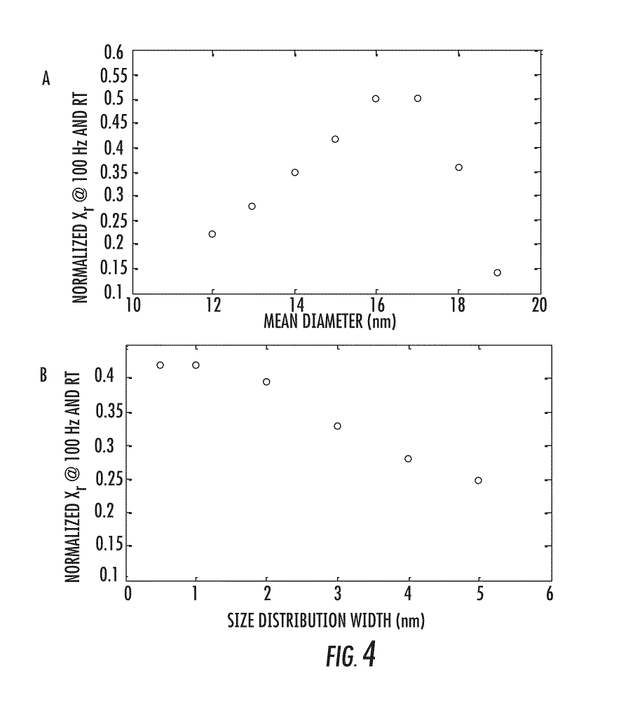

In FORMULA 1 the AC magnetic susceptibility χ can be broken down into a χ_{—real} portion which boosts the signal and needs to maximized for the best sensitivity and a χ_imag imaginary portion which represents energy dissipation and which needs to be minimized. Using these equations the data in FIGS. 1-4 were generated at 25° C. in a liquid state. In FIG. 1A a size distribution is shown for MNPs having a peak diameter of 15 nm and assuming a size distribution of +/−0.5 nm. In FIG. 1B the calculated χ_{—real} portion is shown in the top trace and the calculated χ_imag imaginary portion is shown in the lower trace. It can be seen that the calculated χ_{—real} portion stays essentially constant over the frequency range shown and is at a level of about 0.42. The data of FIGS. 2A and 2B show that as the size distribution increases to +/−2 nm even at the same peak diameter of 15 nm the χ_{—real} value begins to fall especially at frequencies above 100 Hz and the χ_imag imaginary value begins to climb. In FIGS. 3A and 3B the data for a size distribution of +/−5 nm with a peak diameter of 15 nm are shown. Now one sees that the initial value of χ_{—real} is lower and the fall off as the frequency increases is much more dramatic. The value of χ_{—real} at 1000 Hz is about half of that calculated for the 15 nm diameter with a size distribution of +/−0.5 nm. In FIG. 4A the MNP size was varied and the value for the χ_{—real} was plotted after normalization to a frequency of 100 Hz at 25° C. and a size distribution of +/−of 1 nm. This data shows that there is a theoretical AC susceptibility peak normalized value of about 0.5 for this size distribution when the peak diameter is in the range of from 15 to 18 nm. In FIG. 4B the χ_{—real} was calculated assuming a peak diameter of 15 nm and varying the size distribution from +/−0.5 to +/−5 nm with normalization to 100 Hz and 25° C. This data shows that beyond a size distribution of +/−2 nm there is a fairly dramatic fall in the calculated value of the χᵣₑₐₗ.

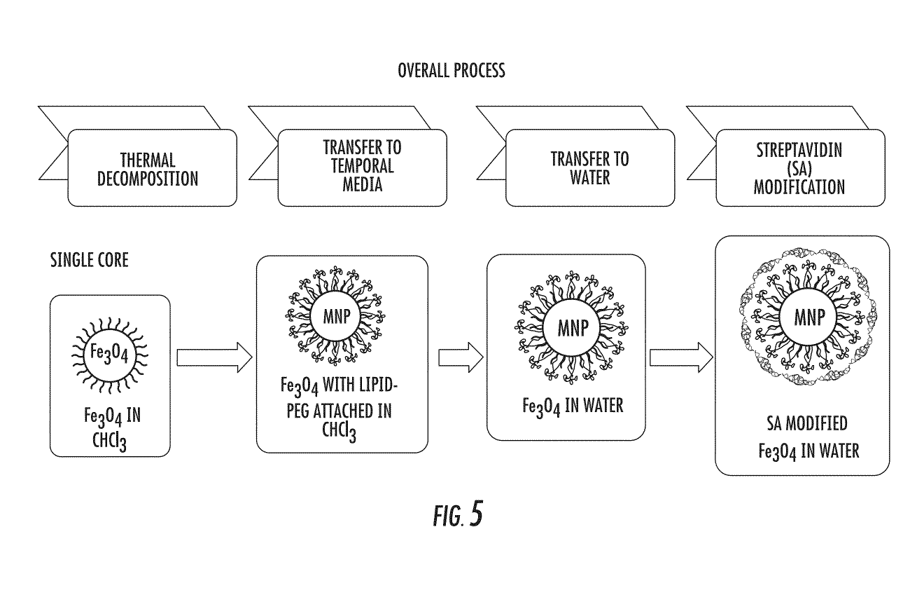

Based on the data of FIGS. 1-4 it can be seen that for ferric oxide MNPs in theory one should use nanoparticles having a size of from 15 to 18 nm and a size distribution of +/−2 nm. The present inventors developed a process for formation of streptavidin (SA) coated MNPs that leads to dramatically better results in GMR sensor applications and holds promise to dramatically improve the usefulness of MNPs. An overview of the process is shown schematically in FIG. 5.

By way of example, in one process according to the present invention iron oxide MNPs having a size of from 10 to 20 nm are prepared according to the following process. In a first step 2 mmol of iron acetylacetonate Fe(acac)3 is combined with 10 mmol 1,2 hexadecanediol, 2 to 4.5 mmol of oleic acid, 2 to 4.5 mmol oleylamine, and 10 mL of trioctylamine. Alternatively, 1-octadecene or ethers such as benzyl ether, dioctyl ether, or diphenyl ether can be used in place of the trioctylamine. The mixture is magnetically stirred under a blanket of a non-reactive gas such as argon or nitrogen and heated to 120° C. for 1 hour. The mixture is next heated to 200° C. for 2 hours. Then the mixture is slowly heated up at a rate of 2° C./minute to a reflux temperature of 350° C. and refluxed for 2 hours. The mixture is then cooled to room temperature, 25° C., by removal of the heat source. Once the mixture reaches room temperature, 25° C., 40 mL of ethanol is added to the mixture and the MNPs are precipitated and separated via centrifugation. The MNPs are then washed several times in ethanol and mixtures of ethanol and chloroform and collected via centrifugation. The MNPs are then suspended in chloroform and stored until used. Alternatively, the MNPs can be stored in other organic solvents such as hexane or toluene. The present inventors have found that adjusting the levels of the oleic acid and oleylamine between 2 to 9 mmol, so that the molar ratio of these surfactants to the molar value of the iron acetylacetonate is varied, independently, from 1:1 to 5:1, one can tune the size of the MNPs created. Ones which show good properties in this application, those having a particle sizes of about 10 to 25 nm, are made by adjusting the levels of the oleic acid and oleylamine to between 2 to 4.5 mmol for 2 mmol of iron acetylacetonate. Thus, preferably the molar ratios of oleic acid and oleylamine to the iron acetylacetonate are, independently, from 1:1 to 2.5:1. These preferred ratios are well below those typically used to create MNPs, namely 3 or greater to 1. The present inventors have found that as the levels of the oleic acid and oleylamine are decreased from 4.5 mmol to 2 mmol the MNPs size also increases. Therefore in the present process preferably the surfactants oleic acid and oleylamine are used during the synthesis of magnetic nanoparticles based on iron oxide. Preferably the molar ratio of oleic acid and the molar ratio of oleylamine to iron acetylacetonate are, independently, from 1:1 to 2.5:1.

As produced the MNPs described above have a coating of the surfactants, oleic acid and oleylamine, on them and are not dispersible in biological or water based solvents. The MNPs as produced are very hydrophobic. To be useful in most desired systems the MNPs must be surface modified to permit them to remain as colloidal suspensions in water or biological based solvent systems and in high salt solutions. As a first step, most commercial MNPs are stored in toluene which must be changed to chloroform so the MNPs in toluene are washed with collection via centrifugation to remove the toluene. To the MNPs addition of trioctylamine is made, after mixing the MNPs are centrifuged, the supernatant is removed and the MNPs are saved. This is done several times to wash the MNPs. After the final wash the MNPs are suspended in chloroform. The MNPs according to the present invention are typically stored in chloroform so this washing step is optional.

The second step is surface modification of the MNPs with functional compounds to make the MNPs more hydrophilic. One process that can be used is to surface modify the MNPs with Lipid-polyethylene glycols (PEG) thereby making the MNPs more hydrophilic. Other surface modification processes can be used as known to those of skill in the art. Useful functional lipid-PEGs for the present invention include the ammonium salts of: 1,2-distearoyl-sn-glycero-3-phosphoethanolamine-N-[methoxy(polyethylene glycol)-2000] (methoxy-PEG); 1,2-distearoyl-sn-glycero-3-phosphoethanolamine-N-[succinyl(polyethylene glycol)-2000] (succinyl-PEG); and 1,2-distearoyl-sn-glycero-3-phosphoethnaolamine-N-[succinyl(polyethylene glycol)-2000] (amine-PEG). The surface can be modified with succinyl-PEG, mixtures of methoxy-PEG and succinyl-PEG, amine-PEG, or mixtures of amine-PEG and methoxy-PEG. In one example process, the functional lipid-PEGs are dissolved in chloroform and then added to the MNPs in chloroform and allowed to react for 5 minutes or more. The functional lipid-PEGs then bind to the surface of the MNPs through a hydrophobic reaction with the surfactants on the surface of the MNPs.

Once the reaction is completed the MNPs are dried under a stream of argon gas. Then the residual chloroform is removed under vacuum at 80° C. The MNPs are then put through several water washes with collection via centrifugation to remove residual lipid-PEG. The water dispersed MNPs with lipid-PEG bound to them can then be subjected to further surface modifications as are known in the art. For experimental purposes in the present invention in one process the MNPs are subjected to streptavidin (SA) modification after the lipid-PEG modification. Streptavidin is a well-known protein biomolecule that has very high affinity for the well-known compound biotin, vitamin B₇. Biotin is also known as vitamin H or coenzyme R. Avidin is another protein biomolecule with high affinity for biotin. The streptavidin-biotin bonding and the avidin-biotin bonding are used in many diagnostic systems, cell sorting processes and immunological assays. When the MNPs have been modified with amine-PEG the streptavidin can be bound to the lipid-PEG functional group using, for example, the well know reaction with 1-ethyl-3(3-dimethylaminopropyl)carbodiimide (EDC). The EDC reaction is used to cause crosslinking between the amine group of the lipid-PEG and a carboxyl group on the streptavidin. Typically the reaction can be carried out at 4° C. overnight. In the case of use of the succinyl-PEG the binding of the streptavidin can be accomplished using the known EDC/Sulfo-NHS reaction wherein Sulfo-NHS is N-hydroxysulfosuccinimide. This reaction is typically carried out at 4° C. overnight or at room temperature for about 2 hours. The unreacted NHS and the EDC are then removed by washing through a magnetic column which retains the MNPs and allows all non-magnetic components to pass through. After several washes the magnetic column is turned off and the MNPs can be collected and dispersed in phosphate buffered saline (PBS). Carrier proteins such as bovine serum albumin (BSA) or the reagent Block ACE can optionally be added to the solution to stabilize the reaction between the MNPs and the streptavidin and between the labeled MNPs and the biotin on the GMRS. In one example, the end result as shown in FIG. 5 is a MNP that has been surface modified with a lipid-PEG and bound streptavidin. The surface modified MNPs are now water dispersible and highly stable in biological systems and buffers. The MNPs according to the present invention can also comprise many other biomolecules as surface modifications. For example, these other biomolecules can include a protein, an antibody, or an enzyme.

As discussed above, one use of MNPs is in sensor applications using GMRS. A GMRS is composed of a laminate array of thin films which alternate non-magnetic layers with magnetic layers. The layers are very thin on the order of about 2 to 30 nm in thickness. In these GMR sensors a giant change in resistivity occurs when the magnetic field changes by small amounts. To test the usefulness of MNPs prepared according to the present invention GMRS signals were evaluated for a variety of commercially available MNPs and for a series of MNPs prepared according to the present invention. For one experiment the GMRS used was initially coated with biotin-BSA so that MNPs coated with streptavidin would bind to the GMRS and thereby create a change in resistance that could be detected. In addition, the results for MNPs prepared according to the present invention were compared to the results obtained using commercially available MNPs conjugated to SA. The MNP sizes for the commercial products were obtained from the manufacturer literature. The particle sizes for MNPs created according to the present invention were estimated by dispersing the MNPs in water or chloroform and then drying them on a grid and using Transmission Electron Microscopy (TEM) counting. Another method of particle size estimation that can be used is the known disc centrifuge technique, which provides for more reliable estimates of particle size. The observed data is reported below in Table 1. The units emu/(Oe mg) is the real value portion of the AC magnetic susceptibility per unit iron oxide weight (mg). The solid state AC susceptibility is obtained using an AC superconducting quantum interference device (AC SQUID) at a frequency of 100 Hz with an amplitude of 5 Oe at 300° Kelvin, which is approximately 27° C., with the magnetic particles in a solid state as required by the methodology. The GMRS signal values after exposure of about 0.1 to 5 mg/ml of test MNPs to the GMRS that are reported are the ratio of the observed value to a standard value using commercially available MACS® SA MNPs. The MACS® SA MNPs are composed of a cluster of about 10 nm or smaller γ-Fe₂O₃ nanoparticles held together by a matrix of dextran. Due to the small size of the γ-Fe₂O₃ nanoparticles, the MACS® SA particles are superparamagnetic, with an overall diameter of about 50 nm and containing about 10% magnetic material. The tested Ocean Nanotech magnetic nanoparticles are also commercially available nanoparticles. Therefore, a GMRS signal value of less than 1 means the test sample had a lower response than the MACS® SA MNPs and value of greater than 1 means the test sample was more responsive than the MACS® SA MNPs.

TABLE 1

| | AC | | | |
|---|---|---|---|---|
| | susceptibility | Particle | Particle | |
| | per weight | size in | size in | GMRS signal |
| MNP ID | emu/(Oe mg) | water nm | CHCl₃ nm | value |
| Modified Ocean | 4.75 ⁽⁻³⁾ | | | 1.06 |
| Nanotech 18 nm | | | | |
| particles | | | | |
| Ocean Nanotech | 1.04 ⁽⁻³⁾ | | | 0.53 |
| 18 nm particles | | | | |
| Ocean Nanotech | 2.77 ⁽⁻⁴⁾ | | | 0.45 |
| 25 nm particles | | | | |
| Inventive MNPs | 7.16 ⁽⁻⁴⁾ | 15.0 | 20.5 | 4.04 |
| sample 1 | | | | |
| Inventive MNPs | 6.76 ⁽⁻⁴⁾ | 14.5 | 18.3 | 2.02 |
| sample 2 | | | | |
| Inventive MNPs | 7.47 ⁽⁻⁴⁾ | 15.8 | 18.0 | 1.83 |
| sample 3 | | | | |
| Inventive MNPs | 7.47 ⁽⁻⁴⁾ | 13.9 | 19.9 | 4.23 |
| sample 4 | | | | |
| Inventive MNPs | 1.24 ⁽⁻³⁾ | 10.9 | | 0.80 |
| sample 5 | | | | |

The data show several interesting trends. In terms of MNPs prepared according to the present invention it is clear that particles in the range of 15 to 20 nm performed better in terms of sensor signal value than did the smaller particle size of 10 nm. This fits with the theoretical data above. This is despite much lower emu/Oe mg values in these larger particles. In addition, the MNPs prepared according to the present invention performed much better than the three commercial Ocean Nanotech samples and when in the size range above 10 nm they performed much better than the standard MACS® SA MNPs. In some cases the MNPs according to the present invention gave 4 times the signal found from the MACS® SA MNPs. This is despite having much lower emu/Oe mg values.

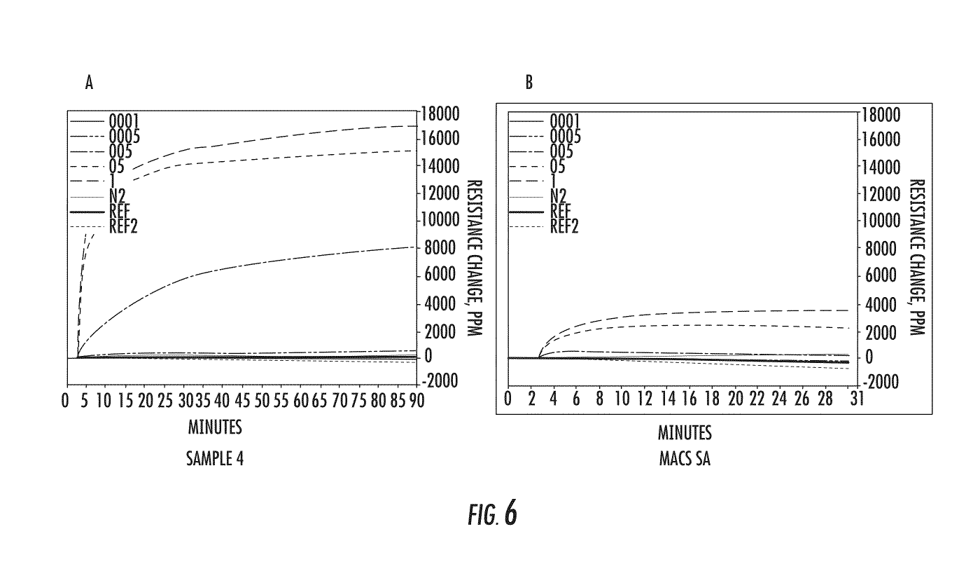

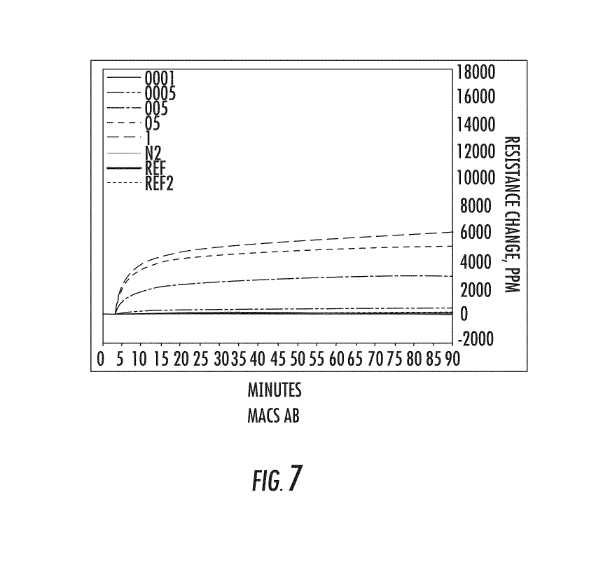

In another analytical experiment the sensitivity of inventive MNPs sample 4 from Table 1 above was compared to several MNPs from Miltenyi Biotec. Specifically, the comparative MNPs were MACS® AB (MNPs conjugated to an antibody to biotin) and MACS SA. The test was to determine the amount of resistance change caused by increasing levels of analyte for GMRS sensors coated with the analyte and then exposed to the test MNPs. In FIG. 6A the MNPs were prepared according to the present invention, sample 4 from Table 1. The curves for REF, REF2 and N2 are control values. The data for the inventive sample 4 shows that 0.0001 mg/ml of analyte was not distinguishable in terms of resistance from the control values for REF, REF2 and N2. All of the curves fall nearly on top of each other. Starting with 0.0005 mg/ml of analyte there begins to be a detectable change in resistance. At a level of 0.005 mg/ml there is a significant change in resistance that reaches a plateau of 8,000 ppm of resistance change after 90 minutes. The ppm value is defined as the resistance change caused by magnetic nanoparticles attached to the sensor with respect to the resistance of the GMRS with no particles attached. At a level of 0.05 mg/ml the plateau is reached at a level of over 15,000 ppm of resistance. Finally, at an analyte level of 1 mg/ml the plateau is reached at about 17,000 ppm of resistance. This is in stark contrast to the results shown in FIG. 6B for the commercial MACS® SA MNPs. In this sample one does not begin to see a detectable resistance change until the analyte level is 0.05 mg/ml, the lower levels and control curves are all superimposed on each other. At an analyte level of 0.05 mg/ml the value plateaus at about 2,200 ppm of resistance. This value is far below the result in FIG. 6A which shows a plateau of over 15,000 ppm of resistance for this level of analyte. The value for 1 mg/ml of analyte in FIG. 6B plateaus at a value of about 3,800 ppm of resistance which is also far below the value of about 17,000 seen in FIG. 6A. Turning to the results shown in FIG. 7 one can see that the commercial MACS® AB is more sensitive than the MACS® SA, but less sensitive than sample 4 prepared according to the present invention. For the MACS® AB one begins to see a detectable change in resistance at an analyte level of 0.005 mg/ml which results in a plateau value of about 2,900 ppm of resistance. An analyte level of 0.05 mg/ml results in a plateau of about 5,000 ppm of resistance. Finally, an analyte level of 1 mg/ml results in a plateau value of 6,000 ppm of resistance. All of the values for MACS® AB are far below the results seen in FIG. 6A for the same levels of analyte using MNPs prepared according to the present invention. The results show that the present MNPs are much more sensitive than commercially available MNPs and offers hope for a dramatic increase in their usefulness.

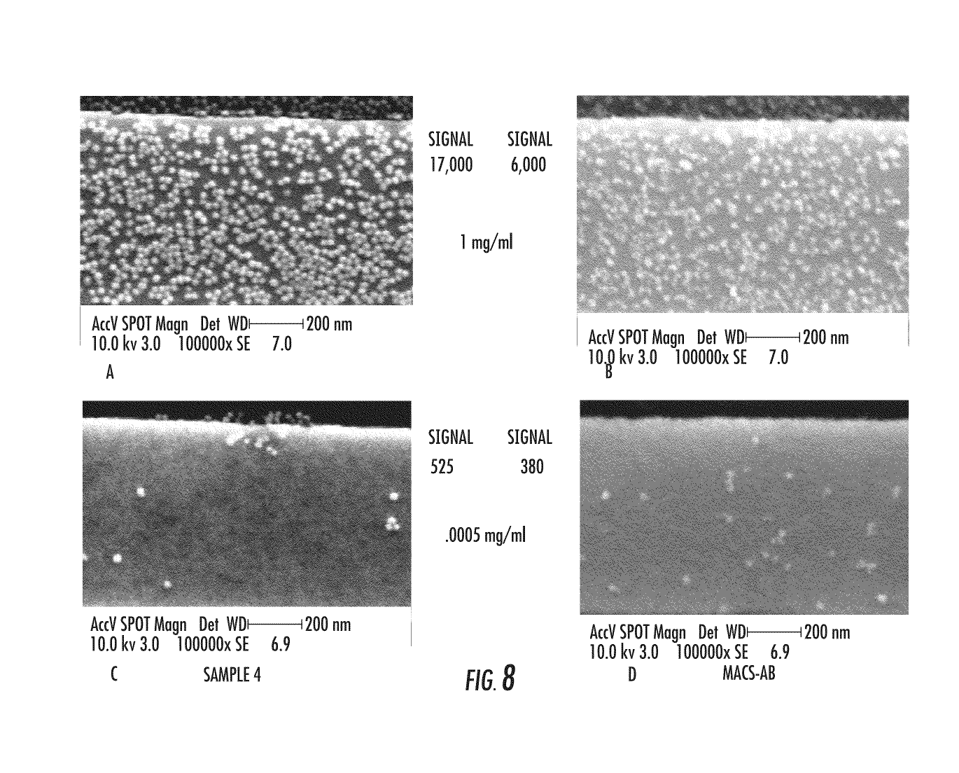

FIGS. 8 A-D show SEM photographs of some of the results shown in FIGS. 6A and FIG. 7. Specifically, FIG. 8A and FIG. 8C show the results from sample 4 at levels of 1 mg/ml of analyte and 0.0005 mg/ml of analyte, respectively. FIG. 8B and FIG. 8D show the results from MACS® AB at levels of 1 mg/ml of analyte and 0.0005 mg/ml of analyte, respectively. Also shown next to each Fig are the actual plateau values for the ppm of resistance. Again these results show the dramatic increase in sensitivity found in MNPs prepared according to the present invention.

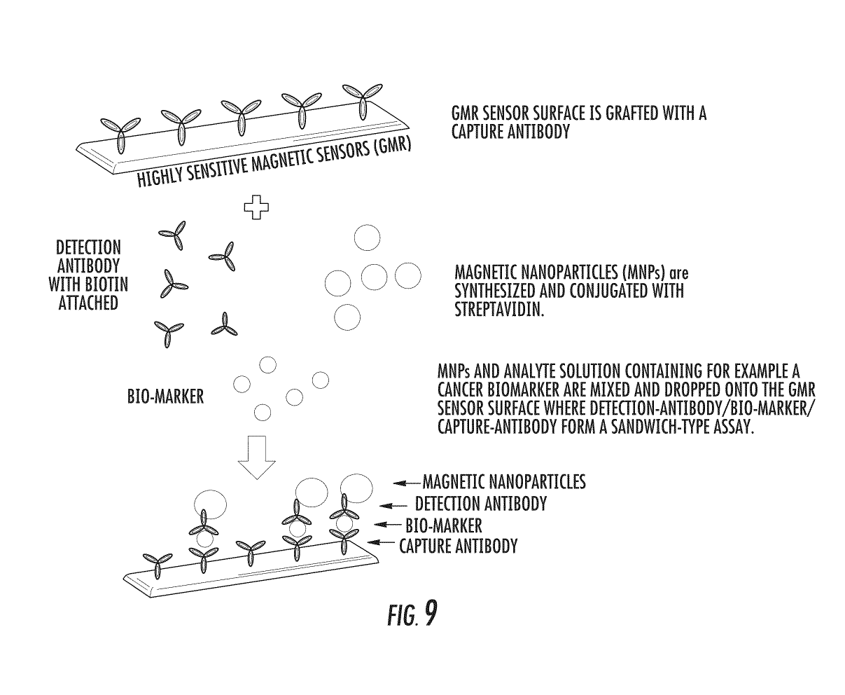

FIG. 9 is a schematic of one way in which the present MNPs will find use in creating a GMRS that is more sensitive than currently available. As shown in the schematic the MNPs can be conjugated with a binder functional group like streptavidin. A detection antibody directed to a cell or analyte of interest includes a functional conjugation to biotin. This permits the MNPs to bind to the detection antibody which are in turn bound to the antigen on the bio-marker. The surface of the GMRS is coated with a capture antibody directed to another antigen on the bio-marker. The system creates a detectable sandwich as shown in the schematic. This technology shows promise for enhancing usefulness of GMRS in many applications because more sensitive GMRS can be prepared. The MNPs prepared according to the present invention will also find use in many areas including applications such as, for example: as contrast agents for magnetic resonance imaging (MRI); magnetic particle imaging (MPI); immunological analytical assays; separation techniques for isolation of cells, proteins, DNA, and RNA; drug delivery using drugs tagged with MNPs and magnetic fields to guide the drugs to the desired target cells or organs; therapeutic treatment of cancer cells or tumors using the MNPs to create hyperthermia in the cancer or tumor cells. In the hyperthermia uses the MNPs are injected into a host and then translocated into the cancer cells or directly injected into the tumor. Then externally applied alternating magnetic fields are used to change the direction of the magnetic fields in the MNPs, then when they are allowed to relax there is heat dissipation and the cancer or tumor cells, which are more temperature sensitive than healthy cells, are preferentially killed. Another use of the MNPs according to the present invention is their use at high levels in fluids to create ferrofluids that can have a tunable viscosity due to aggregation and disaggregation through use of magnetic fields as known in the art.

A series of MNPs of various sizes prepared according to the present invention were compared to commercially available magnetic nanoparticles in terms of their liquid state AC susceptibility. The commercially available magnetic nanoparticles used were: Ocean Nanotech 15 nm particles (ON 15nm); Ocean Nanotech 25 nm particles (ON 25nm); Sigma 20 nm particles (Sigma20 nm); and MACS® AB nanoparticles as described above The sizes of the commercial magnetic nanoparticles are as designated by the manufacturer. The liquid state AC susceptibility of the various magnetic nanoparticles was measured using an AC susceptometer, DynoMag Instrument from Acreo AB, at a frequency of 100 Hz with an amplitude of 5 Oe with the magnetic nanoparticles in water and at a temperature of 25° C. As discussed above, in the present specification and claims the term “in a liquid state” means the magnetic nanoparticles are suspended in a liquid. The preferred liquid is water; however the measured AC susceptibility will be similar in other liquids having a viscosity that is similar to that of water. For purposes of example, other liquids having a sufficiently similar viscosity to water include: phosphate buffered saline, biological buffers, chloroform, toluene, hexane, methanol, and ethanol. If one suspends the magnetic nanoparticles in very high viscosity liquids then there will be a decrease in the AC susceptibility, but this is expected to occur only in very high viscosity liquids. The data generated was then plotted in a series of different ways. In all cases the size of the MNPs in nm was plotted on the X-axis versus the calculated AC susceptibility.

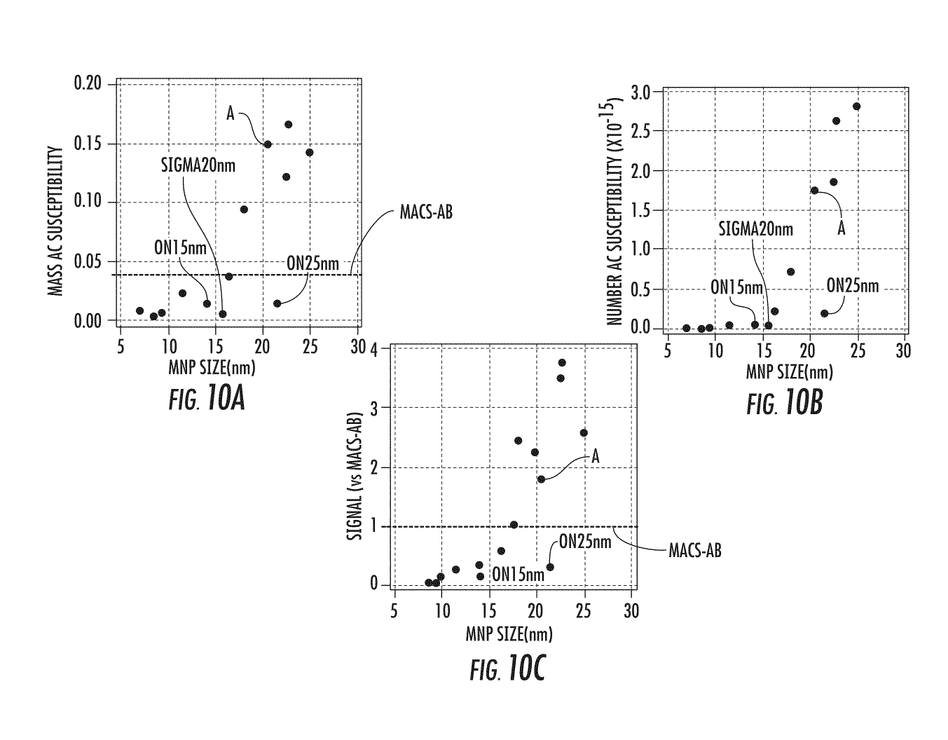

In FIG. 10A the size was plotted versus the mass AC susceptibility, meaning the AC susceptibility per milligram of MNP material. The particle sizes for all the samples were confirmed by Transmission Electron Microscopy (TEM). All the magnetic nanoparticles were transferred to water before the TEM measurements. Note that the observed particle size of the commercially available particles was different from the size stated by the vendors. Several key pieces of information emerge. The other commercial magnetic nanoparticles all had a lower AC susceptibility per milligram of material than the MACS® AB nanoparticles. The AC susceptibility of the two sizes of Ocean Nanotech 15 nm and 25 nm magnetic nanoparticles was the same on a per milligram basis, meaning there was no size dependency for these magnetic nanoparticles. This is in stark contrast to the results using the MNPs according to the present invention. For these inventive MNPs there is a dependence of AC susceptibility per milligram of material on the size of the MNPs. As the size increases the AC susceptibility per milligram also increases to over 3 times higher than the smaller MNP particles. The most size dependence is seen when the MNPs are from about 15 to 25 nm in size.

In FIG. 10B the magnetic particle size in nm was plotted versus the AC susceptibility per nanoparticle, number AC susceptibility. As in FIG. 10A the commercial magnetic nanoparticles did not show much effect of size on the measured AC susceptibility. On the other hand, MNPs according to the present invention showed a clear increase in AC susceptibility per nanoparticle with increasing nanoparticle size. The increase for MNPs according to the present invention was very large, the values achieved in MNPs having a size of 25 nm was about 30 fold higher than those found in MNPs of approximately 10 nm.

In FIG. 10C the relative signal strength compared to MACS® AB nanoparticles is plotted versus particle size. As noted above the other commercial magnetic nanoparticles all provide a signal that is weaker than that of the MACS® AB nanoparticles. The present inventive MNPs show a significant enhanced signal as their size is increased from 15 to 25 nm. The signal is much larger than that seen for the MACS® AB nanoparticles, almost 4 fold higher. The results of FIGS. 10A to 10C show that the present inventive MNPs have a significantly higher AC susceptibility compared to commercial magnetic nanoparticles. The enhanced AC susceptibility results in a significantly higher signal compared to commercially available magnetic nanoparticles.

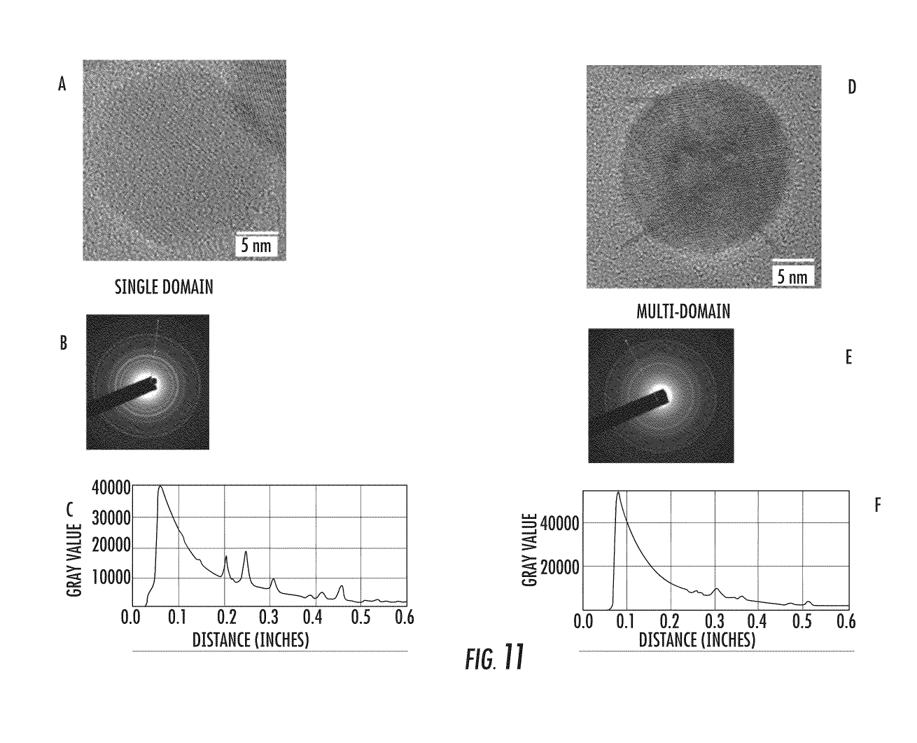

To explore possible reasons for the significantly enhanced AC susceptibility of the present MNPs compared to commercially available magnetic nanoparticles the MNPs were subjected to Transmission Electron Microscopy (TEM) and Selected Area Diffraction (SAD) treatments. A cross-sectional TEM image of a MNP according to the present invention, taken from the preparation designated as A in FIGS. 10A-10C, and an Ocean Nanotech 25 nm magnetic nanoparticle are shown in FIGS. 11A and 11D respectively. The TEM of the inventive MNP shows that it is a single magnetic domain, meaning all of the dipoles are easy to arrange in the same orientation. The Ocean Nanotech magnetic particle TEM shows multiple magnetic domains. The SAD images and their gray value graphs are shown in FIGS. 11B, 11C, 11E and 11F. These results demonstrate that the crystallinity of these two magnetic nanoparticles is very different. The MNP according to the present invention shows clear halo rings and distinct gray value peaks. This is indicative of a highly ordered crystalline structure in the MNPs according to the present invention. On the other hand the results for the Ocean Nanotech particle shows a single fuzzy center with fuzzy outer rings. This confirms the multiple domains seen in the TEM image of FIG. 11D. It is theorized that the highly ordered crystalline structure and single magnetic domain of the present MNPs is partly responsible for their higher AC susceptibility.

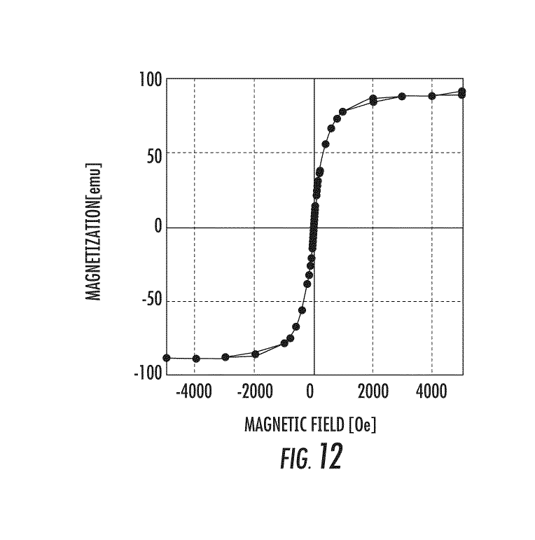

FIG. 12 is a graph of the hysteresis loop for MNPs of sample A in FIGS. 10A-10C according to the present invention. It can be seen that the loop is very narrow, almost superimposed in both directions of magnetism. The field strength required to achieve saturation is much lower than is typical for magnetic nanoparticles. When the magnetic field is small, the slope of the hysteresis loop is very nearly equal to the AC susceptibility. Magnetic reporters like MNPs are typically used at magnetic field strengths sufficient to saturate their magnetization. The results of FIG. 12 show that for the present MNPs this can be achieved at a field strength of 2000 Oe or less. Because of their high AC susceptibility the MNPs according to the present invention can be magnetized to a higher extent at a field strength of about 25 Oe and serve as magnetic reporter particles at these low field strengths. Magnetic particles with high AC susceptibility are great candidates particularly for magnetic field sensors using low magnetic field strengths below 2000 Oe, preferably below 1000 Oe and most preferably below 100 Oe to magnetize the magnetic reporter particles.

In summary, the present inventive MNPs are excellent candidates for highly sensitive magnetic reporter materials. The MNPs are comprised of at least one magnetic material and they can be ferrimagnetic, ferromagnetic, or super paramagnetic in nature. The MNPs have a size of from 10 to 25 nm, preferably 13 to 20 nm and most preferably from 13 to 18 nm. Preferably the size distribution is ±5 nm, more preferably ±2 nm. The MNPs are detectable by GMRS, TMRS, AC SQUID, hall sensors and other magnetic sensors as known to those of skill in the art. The MNPs are detectable in magnetic field strengths of less than 2000 Oe, preferably less than 1000 Oe and most preferably less than 100 Oe. Preferably, the inventive MNPs having a liquid state AC susceptibility per particle at 25° C. that satisfies the following formula:

χ/N≧A(D−13)

Wherein: χ is the AC susceptibility at 100 Hz
N is the number of MNPs
D is the diameter of the nanoparticles in nanometers
A equals

$$\frac{( χ / N )}{D}$$

and ranges in value from 5*10⁻¹⁷ to 2*10⁻¹⁶
   Preferably, the value of χ/_{N} is equal to or greater than 5*10⁻¹⁶. The MNPs according to the present invention have a highly ordered crystalline structure as seen in SAD analysis with clear halo rings and significant gray value peaks showing an ordered structure and a single magnetic domain.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

We claim:

1. Magnetic nanoparticles having a liquid state AC susceptibility per particle at 25° C. that satisfies the following formula:
χ/N≧A(D−13)
Wherein: χ is the AC susceptibility at 100 Hz
N is the number of MNPs
D is the diameter of the nanoparticles in nanometers
A equals
$$\frac{( χ / N )}{D}$$
and ranges in value from 5*10⁻¹⁷ to 2*10⁻¹⁶.

2. The magnetic nanoparticles as recited in claim 1, wherein the value of χ/N is equal to or greater than 5*10⁻¹⁶.

3. The magnetic nanoparticles as recited in claim 1 wherein the magnetic nanoparticles are detectable in a magnetic field strength of 2000 Oe or less.

4. The magnetic nanoparticles as recited in claim 1 wherein the magnetic nanoparticles are detectable in a magnetic field strength of 1000 Oe or less.

5. The magnetic nanoparticles as recited in claim 1 wherein the magnetic nanoparticles are detectable in a magnetic field strength of 100 Oe or less.

6. Iron oxide magnetic nanoparticles having a particle size of from 10 to 25 nanometers with a size distribution of +/−2 nanometers and having a surfactant coating of oleic acid and oleylamine wherein, independently, the molar ratio of oleic acid and the molar ratio of oleylamine to iron oxide are from 1:1 to 2.5:1.

7. The iron oxide magnetic nanoparticles as recited in claim 6, having a particle size of from 13 to 20 nanometers with a size distribution of +/−2 nanometers.

8. The iron oxide magnetic nanoparticles as recited in claim 6, having a particle size of from 13 to 18 nanometers with a size distribution of +/−2 nanometers.

9. The magnetic nanoparticles as recited in claim 1, wherein the magnetic nanoparticles are used as magnetic field reporter particles and are detectable by a magnetic field sensor.

10. The magnetic nanoparticles as recited in claim 9, wherein the magnetic field sensor is one of a giant magnetoresistance (GMR) sensor, a tunnel magnetoresistance (TMR) sensor, a superconducting quantum interference device (SQUID) or a hall sensor.

11. The magnetic nanoparticles as recited in claim 1, wherein said magnetic nanoparticles further comprise at least one biomolecule selected from the group consisting of a protein, an antibody, and an enzyme.

12. The magnetic nanoparticles as recited in claim 1, wherein said magnetic nanoparticles comprise iron ferrite, magnetite, maghemite or a mixture thereof.

13. The magnetic nanoparticles as recited in claim 1 wherein said magnetic nanoparticles are further surface modified with at least one polyethylene glycol.

14. The magnetic nanoparticles as recited in claim 13 wherein said polyethylene glycol comprises at least one of a succinyl-polyethylene glycol, a methoxy-polyethylene glycol, an amine-polyethylene glycol, and mixtures thereof.

15. The magnetic nanoparticles as recited in claim 11 wherein the at least one biomolecule comprises at least one of streptavidin, avidin, biotin, and mixtures thereof.

16. The magnetic nanoparticles as recited in claim 1 wherein each magnetic nanoparticle comprises a single magnetic domain.

17. The magnetic nanoparticles as recited in claim 1 wherein said magnetic nanoparticles have a highly ordered crystalline structure as measured by Selective Area Diffraction analysis.
